(19) United States
(12) Patent Application Publication
(10) Pub. No.: US 2026/0083747 A1
(43) Pub. Date: Mar. 26, 2026

(54) COMPOSITIONS AND METHODS FOR SUPPORTING MITOCHONDRIAL HEALTH

(71) Applicants: Darren LOPEZ, West Valley City, UT (US); Dan SCHMIDT, Salt Lake City, UT (US)

(72) Inventors: Darren LOPEZ, West Valley City, UT (US); Dan SCHMIDT, Salt Lake City, UT (US)

(21) Appl. No.: 18/931,346

(22) Filed: Oct. 30, 2024

Related U.S. Application Data
(60) Provisional application No. 63/698,301, filed on Sep. 24, 2024.

(51) Int. Cl. A61K 31/5415; A61K 9/00; A61K 31/352; A61K 31/439; A61K 31/4745; A61P 3/00

(52) U.S. Cl. CPC A61K 31/5415; A61K 9/0053; A61K 31/352; A61K 31/439; A61K 31/4745; A61P 3/00

(57) ABSTRACT

Methylthioninium salt-containing compositions can include a methylthioninium salt and a second mitochondria-enhancing compound selected from urolithin A having a methylthioninium salt to urolithin A weight ratio from about 1:600 to about 1:4, pyrroloquinoline quinone having a methylthioninium salt to pyrroloquinoline quinone weight ratio from about 1:20 to about 50:1, tesofensine having a methylthioninium salt to tesofensine weight ratio from about 1:2 to about 100:1, or a combination thereof.

The present application claims the benefit of U.S. Provisional Patent Application No. 63/698,301, filed on Sep. 24, 2024, the entirety of which is incorporated herein by reference.

### BACKGROUND

Methylthioninium chloride, referred to conventionally as Methylene Blue, is a synthetic compound often used for treating and/or diagnosing certain medical conditions. It functions similar to hemoglobin, which is a transporter of oxygen. Methylene blue is also a powerful redox agent, which is blue when oxidized and colorless when deoxygenated. In the therapeutic space, methylene blue has been used in the treatment of psychotic disorders, but is not as effective as it could be in treating cognitive and/or other conditions if formulated with other cooperative and/or synergistic compounds.

### DETAILED DESCRIPTION

In accordance with examples of the present disclosure, methylthioninium salt-containing compositions, methods of manufacture, and methods of treatment are provided herein. The compositions can be suitable for oral administration via oral ingestion (swallowing), e.g., capsule, tablet, soft-gel, powder, or liquid, e.g., solution or suspension, and/or via transmucosal oral administration by transmucosal delivery, e.g., buccal, sublabial, sublingual, etc. In some instances, oral delivery may include delivery occurring by oral ingestion with a portion being delivered by transmucosal oral delivery prior to or after swallowing the bulk of the composition, e.g., liquid suspensions may provide some transmucosal delivery due to contact with mucosal tissues within the mouth. These compositions can be suitable for treating various conditions related to mitochondrial health.

In accordance with this, the present disclosure is drawn to methylthioninium salt-containing compositions that can include a methylthioninium salt, e.g., methylene blue, and a second mitochondria-enhancing compound selected from urolithin A having a methylthioninium salt to urolithin A weight ratio from about 1:600 to about 1:4, pyrroloquinoline quinone having a methylthioninium salt to pyrroloquinoline quinone weight ratio from about 1:20 about 50:1, tesofensine having a methylthioninium salt to tesofensine weight ratio from about 1:2 to about 100:1, or a combination thereof. The methylthioninium salt-containing composition can be delivered at a daily dosage of a single oral dosage form, e.g., 1 tablet, or multiple oral dosage forms, e.g., from 2-4 tablets.

In another example, a method of supporting enhanced mitochondrial health in a subject can include orally co-administering a methylthioninium salt and a second mitochondria-enhancing compound to a subject, wherein the second mitochondria-enhancing compound is selected from urolithin A, pyrroloquinoline quinone, tesofensine, or a combination thereof, wherein orally co-administering (on a daily basis) can be at a methylthioninium salt to urolithin A weight ratio from about 1:600 to about 1:4, a methylthioninium salt to pyrroloquinoline quinone weight ratio from about 1:20 to about 50:1, a methylthioninium salt to tesofensine weight ratio from about 1:2 to about 100:1, or a combination thereof.

The methylthioninium salt, in one example, can be a methylthioninium halide compound, such as methylthioninium chloride, e.g., methylene blue. Furthermore, regarding these compositions containing the methylthioninium salt, e.g., methylene blue, and the second mitochondria-enhancing compound, as well as the methods of orally co-administering the methylthioninium salt and the second mitochondria-enhancing compound as set forth herein, in more detail, there are many benefits that can be achieved by utilizing these compositions and/or methods. For example, the methylthioninium salt-containing compositions and methods of the present disclosure can provide a subject with a healthy metabolism through enhanced mitochondrial health, which can assist with maintaining a leaner physique and combatting age-related weight gain. With improved mitochondrial health provided by the different beneficial biological pathways provided by the two compounds of this composition, a subject may expect to experience sustained energy levels without the crashing often associated with certain stimulants. Furthermore, the brain may be sharpened, more focused, and clear, and may also promote better memory retention. This combination can also contribute to improved longevity, as proper mitochondrial function provides cells and tissue that is healthier, leading to a longer life-span with a better quality of life, on average. Furthermore, in some examples, there may be benefits related to weight loss, the curbing of appetite, and/or enhanced cognition as a result of higher brain levels of dopamine, norepinephrine, and/or serotonin, for example. To provide one example, a combination of methylene blue with urolithin A can enhance muscle strength, endurance, and mitochondrial efficiency, e.g., via mitophagy, autophagy, etc. due to the presence of the urolithin A, while the methylthioninium salt can support neuroprotection, cognitive enhancement, overall mitochondrial function, and the like. Other combinations with methylene blue, e.g., pyrroloquinoline quinone (PQQ), tesofensine, etc., can also provide other combined additive effects and in some cases, synergy with respect to various mitochondrial functions and/or biological functions related to mitochondrial health. Thus, the combination of the methylthioninium salt with one or more of the second mitochondria-enhancing compounds described herein can promote healthy mitochondrial function due to the different biological pathways and mechanisms from each of these compounds.

In both the compositions and methods of orally co-administering, the methylthioninium salt-containing composition for delivery can be in an oral dosage form, such as a tablet, capsule, soft-gel, powder, liquid suspension or solution, transmucosal oral formulation, or the like. In formulating the various types of oral dosage forms, the compositions of the present disclosure can include any of a number of formulation additives, such as fillers, excipients, flow agents, etc. These are typically added at minimal concentrations as may be beneficial in preparing the oral dosage form. For example, a liquid suspension may include various additives for stabilizing compounds for suspension and tablets may include a filler, excipient, or flow agent useful for retaining the shape of the individual tablets, etc.

Regarding some of the specific combinations that can be co-formulated or orally co-administered, in one example, the methylthioninium salt and the urolithin A can both be present in a single dosage form, e.g., single table, at a weight ratio from about 1:200 to about 1:6. The methylthioninium salt and the pyrroloquinoline quinone can be present at a weight ratio from about 1:20 to about 30:1. The methylthioninium salt and the tesofensine can be present at a weight ratio from about 1:1 to about 75:1. For example, a single oral dosage form may include from about 1 mg to about 25 mg of the methylthioninium salt. Regarding the second mitochondria-enhancing compound, one or more of the following can be present in a single oral dosage form: from about 100 mg to about 600 mg of urolithin A, from about 0.5 mg to about 20 mg of the pyrroloquinoline quinone, from about 0.25 mg to about 2 mg of the tesofensine, or a combination thereof. The total daily dose can be administered in multiple dosage forms, e.g., from 1-4 tablets, at from about 2 mg to about 50 mg of the methylthioninium salt along with the second mitochondria-enhancing compound, which can be delivered daily at from about 200 mg to about 1200 mg of the urolithin A, from about 1 mg to about 40 mg of the pyrroloquinoline quinone, from about 0.5 mg to about 4 mg of the tesofensine, or a combination thereof.

In further detail, the methylthioninium salt-containing composition can include or be orally co-administered with nicotinamide adenine dinucleotide in the form of NADH, NAD+, or a combination thereof. For example, the compounds in a single oral dosage form can include from about 10 mg to about 100 mg nicotinamide adenine dinucleotide. When delivering, multiple oral dosage forms may be used, e.g., from 1-4 tablets, for a daily dosage of 20 mg to about 200 mg of the nicotinamide adenine dinucleotide. As an example of a methylthioninium salt-containing composition for oral delivery (or separate compositions for oral delivery), the methylthioninium salt and the nicotinamide adenine dinucleotide can be present or orally co-administered at a methylthioninium salt to nicotinamide adenine dinucleotide weight ratio from about 1:100 to about 5:2, and the urolithin A and the nicotinamide adenine dinucleotide can be present or orally co-administered at a urolithin A to nicotinamide adenine dinucleotide weight ratio from about 1:1 to about 60:1. As another example where the urolithin A and the pyrroloquinoline quinone are both present, the urolithin A to pyrroloquinoline quinone weight ratio can be from about 5:1 to about 1200:1.

As used herein, the term “oral dosage form” refers to any of a number of oral delivery compositions or formulations, such as a tablet, capsule, soft-gel, powder, liquid suspension or solution, or transmucosal oral formulation. For example, a single tablet, a single liquid suspension shot, e.g., from 1-4 oz, or the like, refers to the oral dosage form. Furthermore, the term “daily dose” refers to the dosage given to a subject in a given day, either at a single point in time or divided over multiple doses during the day, and thus, can include the use of one or multiple oral dosage forms, e.g., 2 tablets, 3 capsules, 1 soft-gel capsule, etc. The term “daily” does not infer that a subject must take the dosage every single day, but rather refers to how much should be given as a daily dose for any single day. Thus, the methylthioninium salt-containing compositions can be prepared in oral dosage form, e.g., a tablet, a capsule, etc., with higher daily intakes or daily doses being suitable for administration so that some or all of the compounds for administration are available when taken orally as multiple oral dosage forms (of one type or of multiple types of oral dosages forms). Furthermore, it is noted that additional doses can be taken at different times of the day, in some instances, for a total daily dose.

It is also noted that when discussing the methylthioninium salt-containing compositions or any of the related methods herein, discussions of one type of example are considered applicable to other examples whether or not they are explicitly discussed in the context of that example unless expressly indicated otherwise. Thus, for example, when discussing caffeine in the context of one or more of the compositions described herein, such disclosure is also relevant to and directly supported in context of other composition examples as well as any of the methods described herein, and vice versa. Furthermore, for simplicity and illustrative purposes, numerous specific details are set forth in order to provide a thorough understanding of the present disclosure. It will be readily apparent however, that the present disclosure can be practiced without limitation to some of these specific details. In other instances, certain methods, systems, materials, and structures have not been described in detail so as not to obscure the present disclosure.

Furthermore, terms used herein will have their ordinary meaning in the relevant technical field unless specified otherwise. In some instances, there are terms defined more specifically throughout the specification, with a few more general terms included at the end of the specification. These more specifically defined terms have the meaning as described herein.

### Methylthioninium Salt, e.g., Methylthioninium Chloride (Methylene Blue)

Methylthioninium salts, such as methylthioninium chloride (or methylene blue), are synthetic chemical compounds with a crystalline structure. For example, methylthioninium chloride has a specific crystalline structure appearing dark green as a powder at room temperature and blue when mixed in water. The blue color can be lost with time when at rest, e.g., in its reduced state due to lack of interaction with oxygen), but re-emerges as blue when agitated, e.g., in its oxidized state. Because of these unique properties, it has been used diagnostically to distinguish tissues and fluids in a lab setting. Methylthioninium salts can also function similar to hemoglobin, the substance responsible for transporting oxygen to the body's tissues and organs, making it a good candidate for various therapies, though its use orally over the years has led to different levels of success. In further detail, methylthioninium chloride is a formal derivative of phenothiazine, with the hydrated form having three (3) molecules of water per single molecule of the methylthioninium chloride compound. The structure of methylthioninium chloride is shown by way of example in Formula I, as follows:

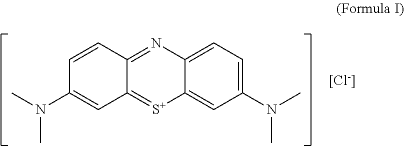

In accordance with the present disclosure, when a methylthioninium salt such as methylthioninium chloride is co-formulated with the second mitochondria-enhancing compound, the impact the combination can have on mitochondrial function and health can be enhanced, particularly when the methylthioninium chloride is given orally at a relatively low dose, and furthermore, is combined with the second mitochondria-enhancing compound at appropriate weight ratios. In some instances, the multiple compounds can exhibit synergy in treating various conditions, including any of a number of cognitive and/or neurological conditions.

It is generally understood that mitochondria are the energy producers of the cells, and often neurodegenerative and psychiatric disorders can lead to mitochondrial damage. The neuroprotective properties of methylthioninium salts can, for example, improve mitochondrial function such that a subject may see improvements in memory retention, cytochrome oxidase activity, ATP generation, etc., thus assisting with the maintenance of neural health and cognitive function. Furthermore, mitochondrial disfunctions can decrease energy transfer to the cells resulting in cellular damage and/or death, e.g., cellular damage can increase neuroinflammation. Furthermore, when mitochondria disfunctions, energy transfers may cease to occur, leading to cellular death. Additionally, toxic build up can occur, leading to oxidative stress and the release of free radicals (and ultimately, loss of neurons). Methylthioninium salt, such as methylene blue, can provide some alternative energy transfer that is effective for protecting neurons by increasing ATP production (which provides energy for the brain via mitochondria respiration). More specifically, as there is a link between methylthioninium salt and neurodegenerative disorders and/or energy metabolism, oral delivery of methylthioninium salt can result in electrons being received from nicotinamide adenine dinucleotide (NADH and/or NAD+, but referred to generally as “NAD”) in the presence of Complex 1 and donate them to Cytochrome C, thus providing an alternative electron transfer pathway. This can result in an increase in oxygen consumption, a decrease in glycolysis, and an increase in glucose uptake enhancing cerebral blood flow. This can be particularly useful for therapeutic uses that increase ATP production after passing through the blood-brain barrier. In accordance with this, in some examples, the compositions of the present disclosure including the methylthioninium salt and the second mitochondria-enhancing compound can be co-formulated or co-administered with nicotinamide adenine dinucleotide, the benefits of which are provided in greater detail hereinafter.

In further detail, methylthioninium salt, particularly when dosed with the mitochondria-enhancing compound that impacts the mitochondria, can act to enhance not only the function of mitochondria, but can act to repair mitochondrial damage and/or provide conditions for mitophagy to occur to degrade damaged mitochondria by autophagy. Examples of mitochondria-enhancing compounds that may provide a secondary mitochondrial benefit include urolithin A, pyrroloquinoline quinone, tesofensine, etc. Furthermore, methylthioninium salt can provide benefits as a superoxide (antioxidant) to reduce or eliminate free radicals that assist in repairing impairments in mitochondrial function and cellular metabolism. In connection with this, by improving mitochondrial function and reducing oxidative damage, methylthioninium salt can contribute to anti-aging in some subjects. For example, methylthioninium salt can bypass Complex I/III activity in the mitochondria, reducing oxidative stress and improving mitochondrial efficiency, which can be beneficial for treating age-related conditions, such as neurodegeneration, memory loss, skin aging, and the like.

In accordance with examples of the present disclosure, the methylthioninium salt-containing compositions of the present disclosure all include methylthioninium chloride, which is a hormetic substance, meaning that it is most effective at lower dosages, and in fact, may be counterproductive if dosed at too high of a level. In accordance with this, the methylthioninium chloride can be present in a single dose (or total daily dose) of one or more of the methylthioninium salt-containing compositions at from about 1 mg to about 50 mg, from about 1 mg to about 25 mg, from about 1 mg to about 20 mg, from about 1 mg to about 15 mg, from about 1 mg to about 12 mg, from about 2 mg to about 20 mg, from about 2 mg to about 15 mg, from about 2 mg to about 12 mg, or from about 2 mg to about 10 mg. In some examples, a single dosage form, e.g., a single tablet, of the methylthioninium salt-containing composition can include about 1 mg to about 25 mg, from about 1 mg to about 20 mg, from about 1 mg to about 12 mg, from about 1 mg to about 6 mg, from about 6 mg to about 12 mg, from about 1 mg to about 3 mg, from about 3 mg to about 6 mg, from about 6 mg to about 9 mg, from about 9 mg to about 12 mg, or from about 12 mg to about 25 mg. These daily dosage (taken at one time or as divided daily dosages) or single dosage form, e.g., single table, capsule, etc., ranges recited above can be used in any of the combination therapies or treatments described by way of example herein, irrespective of the narrower ranges provided for the methylthioninium salt that may be recited hereinafter in the context of more specific examples. Regarding the ratio of the methylthioninium salt to the other compounds for coadministration or in forming the methylthioninium salt-containing compositions may depend on the other compound(s) present, as illustrated by example in greater detail hereinafter.

### Urolithin A

Urolithin A is a postbiotic compound that is instrumental in triggering mitophagy, which is a specific type or autophagy related to cellular recycling. More specifically, urolithin A is a benzo-coumarin (or dibenzo-α-pyrones) compound and is a metabolite of ellagitannin precursors, which can be transformed to urolithin A by certain gut bacteria. In addition to the ellagitannin precursors, Urolithin A has other precursors, such as ellagic acids. More specifically, ellagitannins can be hydrolyzed in the gut to release ellagic acid, which can be further processed by the gut microflora into urolithin A, e.g., via removal of lactone and hydroxyl groups. These precursors are prevalent in various foods and editable plants, such as nuts, e.g., walnuts, pomegranates, berries, e.g., strawberries, raspberries, blackberries, cloudberries, etc., teas, muscadine grapes, and many others.

Even though the precursors are found in natural foods and can be consumed readily, the metabolite urolithin A is not found typically in food sources per se. Furthermore, there are some humans in particular that do not efficiently convert ellagitannins into urolithin A, and other humans may not convert ellagitannins into urolithin A at all. For example, the transformation of ellagic acids into urolithin A depends greatly on the microflora composition in the gut of the subject, which can vary significantly. As fewer and fewer natural foods are being consumed in recent years, the microflora composition of the gut of many individuals may not be optimally established for this transformation. A change in diet may provide an improved microbiome, but genetics may also play a role in the body's ability to make this transformation from precursors to achieving blood serum levels of urolithin A that would be effective in promoting overall mitochondrial health via a healthy level of mitophagy.

In accordance with this, it is believed that less than about 40%-50% of humans can effectively generate urolithin A from its precursors in situ. Thus, for such individuals, supplementation of urolithin A can be highly beneficial, particularly when co-formulated with a methylthioninium salt as described herein, as both together provide a highly effective composition for enhancing the impact that the mitochondria has on the body at a cellular level.

Urolithin A can enter systemic circulation via absorption through the intestines, making it an acceptable compound for oral delivery when dosed at appropriate levels. Urolithin A, as an intact molecule, is shown in Formula II below.

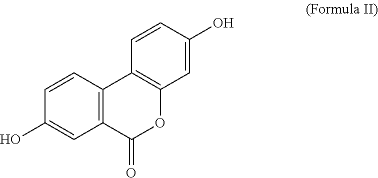 [SMILES: O=c1oc2cc(O)ccc2c2ccc(O)cc12]

Furthermore, when urolithin A is within the body, e.g., the gut, intestinal absorption cells (or enterocytes), systemic circulation, tissue, organs, (hepatocytes such as in the liver), etc., various types of compound transformations may occur in situ, e.g., glucuronidation, methylation, sulfation, etc. As a result, urolithin A has various derivatives, including urolithin A glucuronide, urolithin A sulfate, etc., which can be present in systemic circulation prior to being excreted via the urine. In accordance with this, reference to “urolithin A” herein includes the form of urolithin A shown at Formula II, as well as its salts and derivatives that may be beneficial within the body. Typically, urolithin A as shown in Formula II is delivered orally as described herein, but could be delivered as a salt or as a derivative in some instances.

When co-formulating or orally co-administering with the methylthioninium salt, the methylthioninium salt to urolithin A weight ratio can be from about 1:600 to about 1:4, from about 1:300 to about 1:4, from about 1:200 to about 1:5, from about 1:200 to about 1:6, from about 1:150 to about 1:10, from about 1:100 to about 1:20, or from about 1:80 to about 1:25, either co-formulated or administered as a daily dose (whether by using one oral dosage form, e.g., 1 tablet, or multiple dosage forms, e.g., 2-4 tablets). In terms of daily dosages of the urolithin A with the methylthioninium salt, such dosages can range from about 100 mg to about 1200 mg, from about 200 mg to about 1200 mg, from about 250 mg to about 1000 mg, from about 300 mg to about 750 mg, from about 400 mg to about 900 mg, from about 500 mg to about 750 mg, or from about 200 mg to about 500 mg, for example, e.g., as a single tablet or divided over multiple tablets. For example, if a daily dose to be given to a subject is about 300 mg urolithin A divided over two tablets, each tablet may contain about 150 mg. On the other hand, the 300 mg of urolithin A could be given as a daily dose from a single dosage form, e.g., a single tablet.

As humans age and/or are exposed to various environmental toxins and/or stresses, e.g., EMF, chemical, psychological stress, mitochondrial function tends to decline, leading to increases in chronic disease (due to progressive failure of the mitochondria). In addition to chronic disease, these factors can also lead to reduced energy, slower metabolism, cognitive decline, etc. Repair of the mitochondria, e.g., with mitochondrial DNA damage, such as by processes related to identification of poor cellular function can occur, and when repair is warranted, mitophagy or autophagy may be initiated via cellular reduction along with cellular replacement.

With these factors and others negatively impacting the mitochondria, it has been found that urolithin A provides many benefits, particularly when co-administered in a composition with methylthioninium salts. For example, urolithin A can: i) stimulate the renewal of mitochondria by encouraging replacement of damaged or dysfunctional mitochondria with higher functioning mitochondria; ii) stimulate anti-inflammatory properties and assist with the reducing of inflammation markers associated with chronic disease often exacerbated by inflammation; iii) provide a potent antioxidant to protect cells from damage caused by free radicals; iv) improve muscle strength, growth, and muscle repair (at a cellular level) by influencing health markers and muscle genes associated with mitochondria present in the muscle tissue; and v) positively impact gut health by altering the gut microbiota, improving alpha diversity, restoring colonic barrier function, etc.

To provide one example of the benefits of urolithin on mitochondrial health, the health of muscle tissue can be considered. In accordance with this, the administration of urolithin A can be particularly useful in middle-aged and elderly individuals for muscle health, and furthermore, is sufficiently bioavailable and well tolerated as an oral supplement. Examples of the benefits urolithin A has on muscle tissue include the maintaining or enhancing of muscle endurance, strength, exercise performance, biomarkers of mitochondrial health, and/or overall muscle health. Furthermore, urolithin A can also lower levels of plasma acylcarnitine compounds and C-reactive proteins, which provides evidence for enhanced mitochondrial efficiency and reduced inflammation.

In accordance with this, it has been recognized as described herein that the combination of Urolithin A and with a methylthioninium salt, such as methylene blue, can provide high levels of support for mitochondrial health and muscle function. For example, as mentioned, urolithin A can play a role in promoting mitochondrial mitophagy and can be complemented by the antioxidant properties of methylthioninium salts leading to improved cellular energy production and reduced oxidative stress. As an example, this combination can be particularly beneficial for enhancing physical performance and overall metabolic health when taken at appropriate dosages as described herein.

### Pyrroloquinoline Quinone (PQQ)

Pyrroloquinoline quinone is a compound that is related to the B-vitamin family, which provides strong antioxidant support for mitochondria, which can support cellular energy and assist with maintaining a healthy mind, including supporting a healthy memory. For example, pyrroloquinoline quinone can enhance or support mitochondrial general health as well as growth, and furthermore is neuroprotective against mental function that may be lost due to aging or other conditions. In addition to being effective for mitochondrial health as it relates to mental function, it can also support muscle and other tissue maintenance and growth. Regarding its antioxidant properties, pyrroloquinoline quinone has been reported as being more than two orders of magnitude (>100×) more effective at triggering continuous cycling compared to more common antioxidants, such as vitamin C and various phenolic compounds. In other words, pyrroloquinoline quinone can cycle back between multiple forms, including between a first active form and a second antioxidant form. Cycling back and forth between these two forms, depending on conditions, can provide the ability for performing antioxidant functions as it cycles back and forth for repeated oxidation and reduction reactions. This cycling for repeated antioxidant use provides an excellent way of supporting the central nervous system, e.g., brain, spinal cord, and other nervous system cells or tissue.

In further detail, it is noted that pyrroloquinoline quinone can be administered and/or co-formulated with methylthioninium salts, such as methylene blue, providing a potent antioxidant that supports mitochondrial biogenesis and function. Furthermore, pyrroloquinoline quinone can enhance nicotinamide adenine dinucleotide (NAD) production and use, assisting with mitochondrial efficiency. In further detail, pyrroloquinoline quinone can enhance the housekeeping of the cell with its function supporting mitochondrial mitophagy while the methylthioninium salt can enhance ATP production. As ATP is the source of cellular energy storage and delivery produced by the mitochondria, health implications as it relates to the heart, brain, muscles, and other major organs can benefit greatly from the presence of pyrroloquinoline quinone, particularly when there is some dysfunction, such as the slowing of this functionality as a result of aging. The presence of pyrroloquinoline quinone can assist with supporting the appropriate structure and/or function of the mitochondria.

The structure of pyrroloquinoline quinone is shown by way of example in Formula III, as follows:

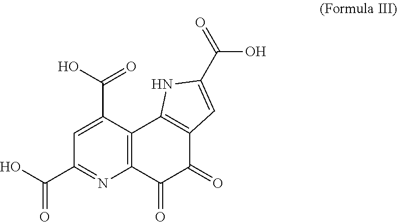 [SMILES: O=C(O)c1cc(C(=O)O)c2c(n1)C(=O)C(=O)c1cc(C(=O)O)[nH]c1-2]

When co-formulating or orally co-administering with the methylthioninium salt, the methylthioninium salt to pyrroloquinoline quinone weight ratio can be from about 1:20 to about 50:1, from about 1:20 to about 30:1, from about 1:10 to about 40:1, from about 1:5 to about 25:1, or from about 1:5 to about 20:1, for example. These weight ratios can be administered orally as a single composition or in separate dosage forms. In terms of individual dosage forms of the pyrroloquinoline quinone with the methylthioninium salt, such dosages of the pyrroloquinoline quinone can range from about 0.25 mg to about 20 mg, from about 1 mg to about 15 mg, from about 0.5 mg to about 10 mg, from about 0.5 mg to about 4 mg, from about 0.75 mg to about 3 mg, or from about 1 mg to about 2.5 mg. Regarding daily dosages to be administered with the methylthioninium salt, the pyrroloquinoline quinone can be administered daily (as a single dose or multiple divided doses) at from about 0.5 mg to about 40 mg, from about 1 mg to about 40 mg, from about 1 mg to about 30 mg, from about 1 mg to about 20 mg, from about 1 mg to about 8 mg, from about 1.5 mg to about 6 mg, or from about 2 mg to about 5 mg as a single dosage form, e.g., tablet, or divided over multiple dosage forms. For example, if a daily dose to be given to a subject includes about 10 mg pyrroloquinoline quinone divided over two tablets, each tablet may contain about 5 mg.

### Tesofensine

Tesofensine is a serotonin-norepinephrine-dopamine reuptake inhibitor (SNDRI). More specifically, tesofensine functions as a triple monoamine reuptake inhibitor, targeting the dopamine transporter (DAT), norepinephrine transporter (NET), and serotonin transporter (SERT). By inhibiting these transporters, tesofensine can increase the levels of dopamine, norepinephrine, and/or serotonin in the brain. This functionality can assist with curbing appetite and enhancing feelings of fullness and satisfaction, even after eating smaller than usual portions of food. Due to this, tesofensine was primarily developed for the treatment of obesity. For example, tesofensine trials have indicated clinical benefits for weight loss, inhibiting the reuptake of neurotransmitters such as serotonin, norepinephrine, and dopamine, which play biological roles in appetite control and metabolism.

In addition to appetite suppression and treatment of obesity, tesofensine may also enhance cognitive function as a result of its impact on the DAT, NET, and SERT. Related to cognitive enhancement is its impact on modulating neuronal activity in the lateral hypothalamus, silencing GABAergic neurons that are involved in hunger signaling. In further detail regarding the impact that tesofensine has on cognition, this compound can indirectly potentiate cholinergic neurotransmission, which has beneficial effects on cognition, particularly in learning and memory in particular. Furthermore, tesofensine can improve mood and reduce anxiety in subjects with depression and anxiety disorders. There may also be a connection between cognitive enhancement and the treatment of obesity, as it can be effective more specifically in treating anxiety and depression in subjects with comorbid depression and anxiety. Tesofensine can also inhibit dopamine transporter proteins, allowing dopamine to stimulate receptors for a more extended period of time. This can provide longer lasting cognitive benefits, and in connection with weight loss, it can enhance the reward system of the brain, providing biological activity causing subjects to feel more satisfied with smaller meals and a reduced desire to snack. In additional detail, tesofensine may provide positive results when treating sexual dysfunction, ADHD, alcohol addiction, sleep disorders, low energy, etc.

The structure of tesofensine is shown by way of example in Formula IV below.

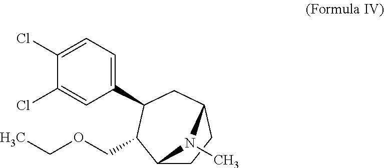 [SMILES: CCOC[C@@H]1[C@@H](c2ccc(Cl)c(Cl)c2)C[C@@H]2CC[C@H]1N2C]

In accordance with the present disclosure, it has been found that co-administering or co-formulating tesofensine with methylthioninium salts, such as methylene blue, can also promote overall mitochondrial health. Methylthioninium salts such as methylene blue, for example, can provide the mitochondrial benefits described previously. Tesofensine, on the other hand, can improve metabolic markers related to insulin sensitivity and blood lipid levels, providing a good option for subjects looking to improve their overall metabolic health. When combined, a methylthioninium salt and tesofensine can offer synergistic benefits in both cognitive and metabolic health. More specifically, tesofensine has the ability to enhance neurotransmitter levels, which can be complimented by the neuroprotective properties of the methylthioninium salt, both leading to improved cognitive function and mood stabilization. Furthermore, the cardiovascular benefits of methylene blue, as an example, can further support the metabolic improvements induced by tesofensine as described above. More specifically, methylene blue as an example can provide neuroprotective and cognitive-enhancing properties to a subject, improving mitochondrial efficiency. This can result in heightened energy, better physical well-being, and improved mental capabilities. Thus, when combined with tesofensine, the methylthioninium salt can actually amplify the cognitive benefits by further enhancing mitochondrial function and reducing oxidative stress.

In some examples, the combination of a methylthioninium salt with tesofensine can be further supplemented by one or more of the other compounds described herein, including urolithin A, pyrroloquinoline quinone, and/or nicotinamide adenine dinucleotide (NAD). In these examples, there may be an additive or even a synergistic benefit to combining pyrroloquinoline quinone with compositions containing the methylthioninium salt and the tesofensine. As mentioned, pyrroloquinoline quinone is a potent antioxidant that supports mitochondrial biogenesis and function. Combining pyrroloquinoline quinone with tesofensine can provide enhancement in energy metabolism, which may also lead to weight loss. Pyrroloquinoline quinone's neuroprotective effects can also complement the administration of the tesofensine as it relates to cognitive benefits. In other examples, there may be an additive or even a synergistic benefit to combining urolithin A with compositions containing the methylthioninium salt and the tesofensine. As mentioned, urolithin A can improve mitochondrial health and muscle function, promoting mitophagy (the process of removing damaged mitochondria), thereby enhancing cellular energy production and overall metabolic health. When combined with tesofensine, Urolithin A can act to enhance overall energy levels and physical performance, which is particularly useful for individuals engaging in physical activity as part of their weight loss regimen.

Thus, as tesofensine offers a range of health benefits, particularly related to weight loss and cognitive enhancement, by modulating key neurotransmitters in the brain, when combined with compounds like methylthioninium salts, pyrroloquinoline quinone, and/or Urolithin A, these benefits can be further amplified. For example, such combinations can offer a comprehensive approach to improving both metabolic and cognitive health. The choice of combination should be tailored to individual health goals and specific therapeutic needs, providing a personalized strategy for enhanced overall well-being. By understanding the mode of action and potential benefits of these combinations, healthcare providers can better tailor treatments to meet the specific needs of their patients.

When co-formulating or orally co-administering with the methylthioninium salt with tesofensine, the methylthioninium salt to tesofensine weight ratio can be from about 1:2 to about 100:1, from about 1:1 to about 75:1, from about 2:1 to about 50:1, from about 4:1 to about 40:1, from about 5:1 to about 30:1, or from about 6:1 to about 20:1, for example. These weight ratios can be administered orally as a single composition or in separate dosage forms. In terms of individual dosage forms including the tesofensine and the methylthioninium salt, such dosages of the tesofensine can range from about 0.125 mg to about 2 mg, from about 0.25 mg to about 1.75 mg, from about 0.4 mg to about 1.75 mg, from about 0.5 mg to about 1.5 mg, or from about 0.5 mg to about 1 mg. Regarding daily doses (single or divided daily dosages) to be administered with the methylthioninium salt, the tesofensine can be administered daily at from about 0.25 mg to about 4 mg, from about 0.5 mg to about 4 mg, from about 0.5 mg to about 3.5 mg, from about 0.8 mg to about 3.5 mg, from about 1 mg to about 3 mg, or from about 1 mg to about 2 mg. For example, if a single daily dose to be given to a subject is about 2 mg divided over two tablets, each tablet may contain about 1 mg. On the other hand, the 2 mg daily dose of tesofensine could be given as a daily dose from a single dosage form, e.g., a single tablet.

### Nicotinamide Adenine Dinucleotide (NAD)

In accordance with examples of the present disclosure, the co-administration or co-formulation of a methylthioninium salt with one or more mitochondria-enhancing compounds such as urolithin A, pyrroloquinoline quinone, and/or tesofensine can further be enhanced by adding a tertiary compound, such as nicotinamide adenine dinucleotide (NAD+ and/or NADH, but referred to generally as NAD). NAD is a versatile compound when present within the body of a subject, and thus, the co-administration or co-formulation of a methylthioninium salt and one of these mitochondria-enhancing compounds can provide further cognitive and/or metabolic benefits. For example, NAD can provide the raw materials for establishing a pathway for the methylthioninium salt to receive electrons from nicotinamide adenine dinucleotide. In some examples when pyrroloquinoline quinone is included as a secondary compound, it can also assist the methylthioninium salt in this regard. To illustrate, as there is a link between methylthioninium salts and energy metabolism, an enhanced functionality of this compound in the body can likewise improve energy. For example, the NAD can provide an alternative electron transfer pathway which may result in an increase in oxygen consumption, a decrease in glycolysis, and an increase in glucose uptake. In some examples, this combination can be particularly useful for producing energy suitable for brain function, as the ATP production enhanced from this combination can occur after passing through the blood-brain barrier.

If nicotinamide adenine dinucleotide (NAD) is included in the composition or co-administered with the methylthioninium salt (and/or other mitochondria-enhancing compound(s)), example methylthioninium salt to nicotinamide adenine dinucleotide weight ratios can be from about 1:100 to about 5:2, from about 1:75 to about 2:1, from about 1:50 to about 3:2, from about 1:50 to about 1:1, or from about 1:25 to about 5:2. Regarding the daily dosage(s) of the nicotinamide adenine dinucleotide to be co-administered with the methylthioninium salt, example daily dosage ranges can be from about 20 mg to about 200 mg, from about 20 mg to about 120 mg, from about 20 mg to about 100 mg, from about 30 mg to about 100 mg, or from about 40 mg to about 100 mg. If divided over multiple oral dosage forms to be taken together or at different times of the day, e.g., 2 tablets, each tablet may include, for example, from about 10 mg to about 100 mg, from about 10 mg to about 75 mg, from about 7.5 mg to about 60 mg, from about 10 mg to about 50 mg, or from about 20 mg to about 50 mg.

### EXAMPLE EMBODIMENTS

In accordance with the disclosure herein, the following examples are illustrative of several embodiments of the present technology.
1. A methylthioninium salt-containing composition, comprising a methylthioninium salt and a second mitochondria-enhancing compound selected from:
urolithin A having a methylthioninium salt to urolithin A weight ratio from about 1:600 to about 1:4;
pyrroloquinoline quinone having a methylthioninium salt to pyrroloquinoline quinone weight ratio from about 1:20 about 50:1;
tesofensine having a methylthioninium salt to tesofensine weight ratio from about 1:2 to about 100:1;
or a combination thereof.
2. The methylthioninium salt-containing composition of example 1, wherein the methylthioninium salt and the urolithin A are present at a weight ratio from about 1:200 to about 1:6.
3. The methylthioninium salt-containing composition of one of examples 1 to 2, wherein the methylthioninium salt and the pyrroloquinoline quinone are present at a weight ratio from about 1:20 to about 30:1.
4. The methylthioninium salt-containing composition of one of examples 1 to 3, wherein the methylthioninium salt and the tesofensine are present at a weight ratio from about 1:1 to about 75:1.
5. The methylthioninium salt-containing composition of one of examples 1 to 4, wherein the urolithin A, the pyrroloquinoline quinone, or both are present.
6. The methylthioninium salt-containing composition of example 5, wherein the tesofensine is present.
7. The methylthioninium salt-containing composition of one of examples 1 to 6, wherein the composition is in an oral dosage form selected from tablet, capsule, soft-gel, powder, liquid suspension or solution, or transmucosal oral formulation.
8. The methylthioninium salt-containing composition of one of examples 1 to 7, wherein a single oral dosage form includes:
about 1 mg to about 25 mg of the methylthioninium salt; and
the second mitochondria-enhancing compound is present and is selected from:
   about 100 mg to about 600 mg of urolithin A,
   about 0.5 mg to about 20 mg of the pyrroloquinoline quinone,
   about 0.25 mg to about 2 mg of the tesofensine, or
   a combination thereof.
9. The methylthioninium salt-containing composition of one of examples 1 to 8, wherein the composition is formulated for a daily dose based on from 1 to 4 oral dosage forms, wherein the 1 to 4 oral dosage forms include a total of from:
about 2 mg to about 40 mg of the methylthioninium salt; and
the second mitochondria-enhancing compound is present and is selected from:
   about 200 mg to about 1200 mg of the urolithin A,
   about 1 mg to about 40 mg of the pyrroloquinoline quinone,
   about 0.5 mg to about 4 mg of the tesofensine, or
   a combination thereof.
10. The methylthioninium salt-containing composition of one of examples 1 to 9, further comprising nicotinamide adenine dinucleotide in the form of NADH, NAD+, or a combination thereof.
11. The methylthioninium salt-containing composition of example 10, wherein a single oral dosage form includes from about 10 mg to about 100 mg of the nicotinamide adenine dinucleotide.
12. The methylthioninium salt-containing composition of one of examples 10 to 11, wherein the composition is formulated for a daily dose based on from 1 to 4 oral dosage forms, wherein the 1 to 4 oral dosage forms include a total of from about 20 mg to about 200 mg of the nicotinamide adenine dinucleotide.
13. The methylthioninium salt-containing composition of one of examples 10 to 12, wherein methylthioninium salt and nicotinamide adenine dinucleotide are present at a methylthioninium salt to nicotinamide adenine dinucleotide weight ratio from about 1:100 to about 5:2, and wherein the urolithin A and nicotinamide adenine dinucleotide are present at a urolithin A to nicotinamide adenine dinucleotide weight ratio from about 1:1 to about 60:1
14. The methylthioninium salt-containing composition of one of examples 1 to 13, wherein the urolithin A and the pyrroloquinoline quinone are both present at a urolithin A to pyrroloquinoline quinone weight ratio from about 5:1 to about 1200:1.
15. The methylthioninium salt-containing composition of one of examples 1 to 14, wherein the methylthioninium salt is in the form of methylthioninium chloride.
16. A method of supporting enhanced mitochondrial health in a subject, comprising orally co-administering a methylthioninium salt and a second mitochondria-enhancing compound to a subject, wherein the second mitochondria-enhancing compound is selected from urolithin A, pyrroloquinoline quinone, tesofensine, or a combination thereof, wherein orally co-administering on a daily basis is at a methylthioninium salt to urolithin A weight ratio from about 1:600 to about 1:4, a methylthioninium salt to pyrroloquinoline quinone weight ratio from about 1:20 to about 50:1, a methylthioninium salt to tesofensine weight ratio from about 1:2 to about 100:1, or a combination thereof.
17. The method of example 16, wherein orally co-administering the methylthioninium salt and the second mitochondria-enhancing compound is via a co-formulated methylthioninium salt-containing composition.
18. The method of one of examples 16 to 17, wherein orally co-administering the methylthioninium salt and the second mitochondria-enhancing compound is via a first oral dosage form containing the methylthioninium salt and a second oral dosage form containing the second mitochondria-enhancing compound.
19. The method of one of examples 16 to 18, wherein the methylthioninium salt-containing composition is dosed on a daily basis with sufficient methylthioninium salt to stimulate ATP production and sufficient urolithin A to recognize or repair mitochondria damage.
20. The method of one of examples 16 to 19, wherein the methylthioninium salt-containing composition is dosed on a daily basis with sufficient methylthioninium salt to stimulate ATP production and sufficient pyrroloquinoline quinone to enhance:
nicotinamide adenine dinucleotide production,
mitochondrial mitophagy,
or a combination thereof.
21. The method of one of examples 16 to 20, wherein the methylthioninium salt-containing composition is dosed on a daily basis with sufficient methylthioninium salt to stimulate ATP production and sufficient tesofensine to enhance:
cognition due to increased levels of dopamine, norepinephrine, serotonin, or a combination thereof in the brain,
feelings of fullness and satisfaction by curbing appetite, or a combination thereof.

22. The method of one of examples 16 to 21, wherein orally co-administering includes orally co-administering on a daily basis:
about 2 mg to about 50 mg of the methylthioninium salt; and
the second mitochondria-enhancing compound including:
   about 200 mg to about 1200 mg of the urolithin A,
   about 1 mg to about 40 mg of the pyrroloquinoline quinone,
   about 0.5 mg to about 4 mg of the tesofensine, or
   a combination thereof.
23. The method of one of examples 16 to 22, wherein orally co-administering includes orally co-administering the methylthioninium salt and the second mitochondria-enhancing compound to the subject on a daily basis for at least about one week.
24. The method of one of examples 16 to 23, wherein orally co-administering includes co-administering the methylthioninium salt and the second mitochondria-enhancing compound the subject on a daily basis for at least about one month.
25. The method of one of examples 16 to 24, wherein orally co-administering further includes orally co-administering nicotinamide adenine dinucleotide in the form of NADH, NAD+, or a combination thereof.
26. The method of example 25, wherein the nicotinamide adenine dinucleotide is orally co-administered at from about 10 mg to about 200 mg.
27. The method of one of examples 16 to 26, wherein the methylthioninium salt is in the form of methylthioninium chloride.

### Definitions

In describing and claiming the present technology, the following terminology will be used.

The singular forms “a,” “an,” and “the” include plural references unless the context clearly dictates otherwise. Thus, for example, reference to “an additive” includes reference to one or more of such components, “a suspension” includes reference to one or more of such suspensions, and “the mixing” refers to one or more of such mixing steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, “about” refers to a degree of deviation based on experimental error typical for the particular property identified. The latitude provided by the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” expressly includes “exactly,” consistent with the discussion below regarding ranges and numerical data.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and 200, but also to include individual sizes such as 2, 3, 4, and sub-ranges such as 10 to 50, 20 to 100, etc.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the terms “treatment” or “treating” of a condition and/or a disease in a mammal means preventing the condition or disease, that is, avoiding any clinical symptoms of the disease; inhibiting the condition or disease, that is, arresting the development or progression of clinical symptoms; and/or relieving the condition or disease, that is, causing the regression of clinical symptoms and/or healing. In further detail, “treating” can include oral administration of any of the methylthioninium salt-containing compounds described herein to heal a subject of a condition or disease, reduce or ameliorate symptoms of a condition or disease, prevent or protect a subject from experiencing or relapsing relative to a condition or disease, causing a condition or disease to regress or retreat, etc. “Oral” routes of administration include oral ingestion (swallowing) via capsule, tablet, soft-gel, powder, or liquid, e.g., solution or suspension, and/or via transmucosal oral administration, e.g., buccal, sublabial, sublingual, etc. In some instances, some transmucosal oral delivery may occur prior to or after swallowing the bulk of the oral dosage form.

### EXAMPLES

The following examples illustrate embodiments of the disclosure that are presently best known. However, it is to be understood that the following are only exemplary or illustrative of the application of the principles of the present technology. Numerous modifications and alternative compositions, methods, and systems may be devised by those skilled in the art without departing from the spirit and scope of the present disclosure. The appended claims are intended to cover such modifications and arrangements. Thus, while the present disclosure has been described herein with particularity, the following examples provide further detail in connection with what are presently deemed to be practical embodiments of the disclosure.

### Example 1—Preparation of Methylthioninium Salt-Containing Composition 1 (MB1)

A methylthioninium salt and urolithin A-containing composition (MB1) is prepared by admixing pulverized granules/powder of methylthioninium chloride and urolithin A at about a 1:16.6 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 15 mg methylthioninium chloride and about 250 mg urolithin A. In some examples, when forming compressed tablets, the tablets may be coated with a wax or polymer applied thereto. It is noted that the various components can be combined in liquid form and dried, or can be delivered as a liquid suspension.

### Example 2—Preparation of Methylthioninium Salt-Containing Composition 2 (MB2)

A methylthioninium salt and urolithin A-containing composition (MB2) is prepared by admixing pulverized granules/powder of methylthioninium chloride and urolithin A at about a 1:70 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 5 mg methylthioninium chloride and about 350 mg urolithin A. In some examples, when forming compressed tablets, the tablets may be coated with a wax or polymer applied thereto. It is noted that the various components can be combined in liquid form and dried, or can be delivered as a liquid suspension.

### Example 3—Preparation of Methylthioninium Salt-Containing Composition 3 (MB3)

A methylthioninium salt and urolithin A-containing composition (MB3) is prepared by admixing pulverized granules/powder of methylthioninium chloride and urolithin A at about a 1:50 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 5 mg methylthioninium chloride and about 250 mg urolithin A. In some examples, when forming compressed tablets, the tablets may be coated with a wax or polymer applied thereto. It is noted that the various components can be combined in liquid form and dried, or can be delivered as a liquid suspension.

### Example 4—Preparation of Methylthioninium Salt-Containing Composition 4 (MB4)

A methylthioninium salt and urolithin A-containing composition (MB4) is prepared by admixing pulverized granules/powder of methylthioninium chloride, urolithin A, and pyrroloquinoline quinone at about a 1:25:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 250 mg urolithin A, about 10 mg methylthioninium chloride, and about 10 mg of pyrroloquinoline quinone. In some examples, when forming compressed tablets, the tablets may be coated with a wax or polymer applied thereto. It is noted that the various components can be combined in liquid form and dried, or can be delivered as a liquid suspension.

### Example 5—Preparation of Methylthioninium Salt-Containing Composition 5 (MB5)

A methylthioninium salt and urolithin A-containing composition (MB5) is prepared by admixing pulverized granules/powder of methylthioninium chloride, urolithin A, and nicotinamide adenine dinucleotide at about a 1:75:10 weight ratio. In further detail, other formulation excipients are included for purposes of forming tablets and/or a granule blend for loading into capsules. The granules are then compressed into tablets (or loaded into capsules), with each oral dose, divided over 2 tablets/capsules, including about 10 mg methylthioninium chloride, about 750 mg urolithin A, and about 100 mg of nicotinamide adenine dinucleotide. Thus, each tablet/capsule would have half (½) of the milligram content of the full oral dose. In some examples, when forming compressed tablets, the tablets may be coated with a wax or polymer applied thereto. It is noted that the various components can be combined in liquid form and dried, or can be delivered as a liquid suspension.

### Example 6—Preparation of Methylthioninium Salt-Containing Composition 6 (MB6)

A methylthioninium salt-containing composition (MB6) is prepared by admixing pulverized granules/powder of methylthioninium chloride, urolithin A, nicotinamide adenine dinucleotide, and pyrroloquinoline quinone at about a 2:200:40:1 weight ratio. In further detail, other formulation excipients are included for purposes of forming tablets and/or a granule blend for loading into capsules. The granules are then compressed into tablets (or loaded into capsules), with each oral dose, divided over 2 tablets or capsules, including about 5 mg methylthioninium chloride, about 500 mg urolithin A, about 100 mg nicotinamide adenine dinucleotide, and about 2.5 mg of pyrroloquinoline quinone. Thus, each tablet/capsule would have half (½) of the milligram content of the full oral dose. In some examples, when forming compressed tablets, the tablets may be coated with a wax or polymer applied thereto. It is noted that the various components can be combined in liquid form and dried, or can be delivered as a liquid suspension.

### Example 7—Preparation of Methylthioninium Salt-Containing Composition 7 (MB7)

A methylthioninium salt-containing composition (MB7) is prepared by admixing pulverized granules/powder of methylthioninium chloride and pyrroloquinoline quinone at about a 1:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 10 mg methylthioninium chloride and about 10 mg of pyrroloquinoline quinone. In some examples, when forming compressed tablets, the tablets may be coated with a wax or polymer applied thereto. It is noted that the various components can be combined in liquid form and dried, or can be delivered as a liquid suspension.

### Example 8—Preparation of Methylthioninium Salt-Containing Composition 8 (MB8)

A methylthioninium salt-containing composition (MB8) is prepared by admixing pulverized granules/powder of methylthioninium chloride and tesofensine at about a 30:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 15 mg methylthioninium chloride and about 0.5 mg of tesofensine. A recommended daily dosage for this composition can be two tablets per day for a total of 10 mg methylthioninium chloride and 1 mg tesofensine. In some examples, when forming compressed tablets, the tablets may be coated with a wax or polymer applied thereto. It is noted that the various components can be combined in liquid form and dried, or can be delivered as a liquid suspension.

### Example 9—Preparation of Methylthioninium Salt-Containing Composition 9 (MB9)

A methylthioninium salt-containing composition (MB9) is prepared by admixing pulverized granules/powder of methylthioninium chloride, pyrroloquinoline quinone, and tesofensine at about a 15:20:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 7.5 mg methylthioninium chloride, about 10 mg of pyrroloquinoline quinone, and about 0.5 mg of tesofensine. A recommended daily dosage for this composition can be two tablets per day for a total of 15 mg methylthioninium chloride, 20 mg of pyrroloquinoline quinone, and 1 mg tesofensine. In some examples, when forming compressed tablets, the tablets may be coated with a wax or polymer applied thereto. It is noted that the various components can be combined in liquid form and dried, or can be delivered as a liquid suspension.

### Example 10—Preparation of Methylthioninium Salt-Containing Composition 10 (MB10)

A methylthioninium salt-containing composition (MB 10) is prepared by admixing pulverized granules/powder of methylthioninium chloride, urolithin A, pyrroloquinoline quinone, and tesofensine at about a 10:300:4:1 weight ratio. In further detail, other formulation excipients are included for purposes of forming tablets and/or a granule blend for loading into capsules. The granules are then compressed into tablets (or loaded into capsules), with each oral dose in one (1) tablet or capsule, including about 5 mg methylthioninium chloride, about 150 mg urolithin A, about 2 mg of pyrroloquinoline quinone, and about 0.5 mg of tesofensine. A recommended daily dosage for this composition can be one or two tablets per day. For one tablet, the total daily dosage for each compound can be 5 mg methylthioninium chloride, 150 mg of urolithin A, 2 mg of pyrroloquinoline quinone, and 0.5 mg tesofensine. For two tablets, the total daily dosage for each compound can be 10 mg methylthioninium chloride, 300 mg of urolithin A, 4 mg of pyrroloquinoline quinone, and 1 mg tesofensine. In some examples, when forming compressed tablets, the tablets may be coated with a wax or polymer applied thereto. It is noted that the various components can be combined in liquid form and dried, or can be delivered as a liquid suspension. In some examples, the methylthioninium salt containing composition (MB10) can be co-formulated to also include 250 mg of nicotinamide adenine dinucleotide per tablet.

### Example 11—Oral Administration to Subject 1

A 44 year old male with reduced mood and energy, also having a strong appetite was given the methylthioninium salt-containing compound of Example 1 (MB1). After a period of 3 weeks with daily dosages of 30 mg methylene blue and 500 mg urolithin A (active ingredients as outlined in the composition example, but at 2 tablets per day), the subject reported significant improvement in overall mood and energy, with a more noticeable decrease in appetite.

### Example 12—Oral Administration to Subject 2

A 60 year old male seeking improved stamina and decreased appetite cravings for processed foods was given the methylthioninium salt-containing compound of Example 1 (MB1). After 30 days with daily dosages of 30 mg methylene blue and 500 mg urolithin A (active ingredients as outlined in the composition example, but at 2 tablets per day), the subject reported a curbed appetite while eating and more efficient digestion and better body function, reporting a 5 pound weight loss in the 30 days without any change in daily activity or exercise.

### Example 13—Oral Administration to Subject 3

A 72 year old male was given the methylthioninium salt-containing compound of Example 1 (MB1) and reported results after about 2 weeks. The subject took daily dosages of 30 mg methylene blue and 500 mg urolithin A (active ingredients as outlined in the composition example, but at 2 tablet per day), and reported no adverse effects switching from a methylene blue compound without the urolithin A present, and furthermore, an improvement was noted due to the addition of the urolithin A. More specifically, the subject reported very calm and clear cognition that has been sustained. Furthermore, the subject reported that there was no discomfort nor any noticeable side effects of any kind, indicating he felt 10 years younger, e.g., as in his early 60s.

### Example 14—Oral Administration to Subject 4

A 54 year old woman looking to boost her energy level was given the methylthioninium salt-containing compound of Example 1 (MB1) with daily dosages of 30 mg methylene blue and 500 mg urolithin A (active ingredients as outlined in the composition example, but at 2 tablets per day). The subject reported “incredible” results with much more energy, improving her exercise and biking routines. She reported being able to push her workouts further along with a significant reduction in appetite, making healthy food choices easier without feeling hungry or food deprived. Less time to go to sleep and waking up well rested and refreshed was also noted.

### Example 15—Oral Administration to Subject 5

A 56 year old male skeptic of supplements who prioritized fitness (reporting 10% body fat) was given the methylthioninium salt-containing compound of Example 1 (MB8) and reported a six (6) pound weight loss and a positive impact on workout sessions. The subject also reported a surge in energy during exercise, making completing an exercise session easier. The subject also reported enhanced reserve of strength and stamina. The subject took daily dosages of 30 mg methylene blue and 1 mg tesofensine (active ingredients as outlined in the composition example, but at 2 tablets per day).

It is to be understood that the above-referenced arrangements are illustrative of the application for the principles of the present disclosure. Thus, while the present technology has been described herein in connection with the exemplary embodiments, it will be apparent to those of ordinary skill in the art that numerous modifications and alternative arrangements can be made without departing from the principles and concepts of the disclosure as set forth in the claims.

1. A methylthioninium salt-containing composition, comprising an oral dosage form of a methylthioninium salt and a second mitochondria-enhancing compound selected from:
urolithin A having a methylthioninium salt to urolithin A weight ratio from about 1:600 to about 1:4;
pyrroloquinoline quinone having a methylthioninium salt to pyrroloquinoline quinone weight ratio from about 1:20 about 50:1;
tesofensine having a methylthioninium salt to tesofensine weight ratio from about 1:2 to about 100:1;
or a combination thereof.

2. The methylthioninium salt-containing composition of claim 1, wherein:
the methylthioninium salt and the urolithin A are present at a weight ratio from about 1:200 to about 1:6;
the methylthioninium salt and the pyrroloquinoline quinone are present at a weight ratio from about 1:20 to about 30:1; or
a combination thereof.

3. (canceled)

4. The methylthioninium salt-containing composition of claim 1, wherein the methylthioninium salt and the tesofensine are both present at a weight ratio from about 1:1 to about 75:1.

5. The methylthioninium salt-containing composition of claim 1, wherein the urolithin A, the pyrroloquinoline quinone, or both are present.

6. The methylthioninium salt-containing composition of claim 5, wherein the tesofensine is present.

7. (canceled)

8. The methylthioninium salt-containing composition of claim 1, wherein a single oral dosage form includes:
about 1 mg to about 25 mg of the methylthioninium salt; and
the second mitochondria-enhancing compound is present and is selected from:
about 100 mg to about 600 mg of urolithin A,
about 0.5 mg to about 20 mg of the pyrroloquinoline quinone,
about 0.25 mg to about 2 mg of the tesofensine, or
a combination thereof.

9. The methylthioninium salt-containing composition of claim 1, wherein the composition is formulated for a daily dose based on from 1 to 4 oral dosage forms, wherein the 1 to 4 oral dosage forms include a total of from:
about 2 mg to about 50 mg of the methylthioninium salt; and
the second mitochondria-enhancing compound is present and is selected from:
about 200 mg to about 1200 mg of the urolithin A,
about 1 mg to about 40 mg of the pyrroloquinoline quinone,
about 0.5 mg to about 4 mg of the tesofensine, or
a combination thereof.

10. The methylthioninium salt-containing composition of claim 1, further comprising nicotinamide adenine dinucleotide in the form of NADH, NAD+, or a combination thereof, wherein the composition is formulated as a single oral dosage form including from about 10 mg to about 100 mg of the nicotinamide adenine dinucleotide or wherein the composition is formulated for a daily dose based on from 1 to 4 oral dosage forms providing a total of from about 20 mg to about 200 mg of the nicotinamide adenine dinucleotide.

11-12. (canceled)

13. The methylthioninium salt-containing composition of claim 10, wherein methylthioninium salt and nicotinamide adenine dinucleotide are present at a methylthioninium salt to nicotinamide adenine dinucleotide weight ratio from about 1:100 to about 5:2, and wherein the urolithin A and nicotinamide adenine dinucleotide are present at a urolithin A to nicotinamide adenine dinucleotide weight ratio from about 1:1 to about 60:1.

14. The methylthioninium salt-containing composition of claim 1, wherein the urolithin A and the pyrroloquinoline quinone are both present at a urolithin A to pyrroloquinoline quinone weight ratio from about 5:1 to about 1200:1.

15. The methylthioninium salt-containing composition of claim 1, wherein the methylthioninium salt is in the form of methylthioninium chloride.

16. A method of supporting enhanced mitochondrial health in a subject, comprising orally co-administering a methylthioninium salt and a second mitochondria-enhancing compound to a subject, wherein the second mitochondria-enhancing compound is selected from urolithin A, pyrroloquinoline quinone, tesofensine, or a combination thereof, wherein orally co-administering on a daily basis is at a methylthioninium salt to urolithin A weight ratio from about 1:600 to about 1:4, a methylthioninium salt to pyrroloquinoline quinone weight ratio from about 1:20 to about 50:1, a methylthioninium salt to tesofensine weight ratio from about 1:2 to about 100:1, or a combination thereof.

17. The method of claim 16, wherein orally co-administering the methylthioninium salt and the second mitochondria-enhancing compound is via a co-formulated methylthioninium salt-containing composition.

18. The method of claim 16, wherein orally co-administering the methylthioninium salt and the second mitochondria-enhancing compound is via a first oral dosage form containing the methylthioninium salt and a second oral dosage form containing the second mitochondria-enhancing compound.

19. The method of claim 16, wherein the methylthioninium salt-containing composition is dosed on a daily basis with sufficient methylthioninium salt to stimulate ATP production and sufficient urolithin A to recognize or repair mitochondria damage.

20. The method of claim 16, wherein the methylthioninium salt-containing composition is dosed on a daily basis with sufficient methylthioninium salt to stimulate ATP production and sufficient pyrroloquinoline quinone to enhance:
nicotinamide adenine dinucleotide production,
mitochondrial mitophagy,
or a combination thereof.

21. The method of claim 16, wherein the methylthioninium salt-containing composition is dosed on a daily basis with sufficient methylthioninium salt to stimulate ATP production and sufficient tesofensine to enhance:
cognition due to increased levels of dopamine, norepinephrine, serotonin, or a combination thereof in the brain,
feelings of fullness and satisfaction by curbing appetite, or
a combination thereof.

22. The method of claim 16, wherein orally co-administering includes orally co-administering on a daily basis:
about 2 mg to about 40 mg of the methylthioninium salt; and
the second mitochondria-enhancing compound including:
about 200 mg to about 1200 mg of the urolithin A,
about 1 mg to about 40 mg of the pyrroloquinoline quinone,
about 0.5 mg to about 4 mg of the tesofensine, or
a combination thereof.

23. The method of claim 16, wherein orally co-administering includes orally co-administering the methylthioninium salt and the second mitochondria-enhancing compound to the subject on a daily basis for at least about one week.

24. (canceled)

25. The method of claim 16, wherein orally co-administering further includes orally co-administering nicotinamide adenine dinucleotide in the form of NADH, NAD+, or a combination thereof at a daily dose of from about 20 mg to about 200 mg.

26-27. (canceled)
